(19) United States
(12) Patent Application Publication
(10) Pub. No.: US 2026/0369380 A1
(43) Pub. Date: Nov. 19, 2026

(54) INFORMATION PROCESSING DEVICE, INFORMATION PROCESSING METHOD, AND COMPUTER PROGRAM

(71) Applicant: National University Corporation Tokai National Higher Education and Research System, Nagoya-shi, Aichi (JP)

(72) Inventors: Taiki FURUKAWA, Nagoya-shi, Aichi (JP); Shintaro OYAMA, Nagoya-shi, Aichi (JP)

(21) Appl. No.: 19/135,449

(22) PCT Filed: Jul. 2, 2024
(86) PCT No.: PCT/JP2024/023924

§ 371 Date: Jun. 4, 2025

(30) Foreign Application Priority Data
Jul. 3, 2023 (JP) 2023-109008

(51) Int. Cl. G16H 20/30

(52) U.S. Cl. CPC G16H 20/30

(57) ABSTRACT

An information processing device includes a recommended rehabilitation processing unit, an actual-results information acquisition unit, and a nudge processing unit. The recommended rehabilitation processing unit acquires recommended rehabilitation information indicating recommended rehabilitation for a patient suffering from a disease and outputs the recommended rehabilitation in a form perceivable by the patient. The actual-results information acquisition unit acquires rehabilitation actual-results information indicating a status of performance of rehabilitation by the patient. The nudge processing unit executes, based on the recommended rehabilitation information and the rehabilitation actual-results information, nudge processing that prompts performance of rehabilitation that is appropriate to the patient in terms of behavioral economics.

### TECHNICAL FIELD

The technique disclosed herein relates to information processing on diseases.

### BACKGROUND ART

Interstitial lung disease is a collective term for chronic progressive fibrotic lung diseases. Acute exacerbation of interstitial lung disease is a disease condition in which the disease condition rapidly worsens within one month and has an extremely poor prognosis with an in-hospital mortality rate of about 50%.

In the case of interstitial lung disease, it is expected that patient's daily performance of rehabilitation (hereinafter also referred to as “rehab” for short) improves the prognosis and the QoL (Quality of Life). However, the number of medical workers who can give guidance for rehabilitation is limited, and it is up to patients to perform rehabilitation daily at home; therefore, in many cases, rehabilitation is not continuously performed. High-intensity and frequent rehabilitation is not always desirable, and it is preferable to make adjustments, for example, the intensity is weakened or the number of times is reduced in accordance with the state of the patient.

In the related art, a technique is disclosed in which, in order to induce a user's motivation to engage in rehabilitation, biological information of the user is acquired by a sensor, the state of the user is estimated based on the acquired biological information, and the display is performed in accordance with the estimated state of the user (for example, see Patent Literature 1).

### CITATION LIST

### Patent Literature

Patent Literature 1: Japanese Unexamined Patent Application Publication No. 2021-33442

### SUMMARY OF INVENTION

### Technical Problem

In the above-described related art, there is room for improvement in effectively prompting the patient to perform appropriate rehabilitation. Such an issue is not limited to rehabilitation of interstitial lung disease, but is common to rehabilitation of general diseases.

This specification discloses a technique capable of solving the above-described issue.

### Solution to Problem

The technique disclosed herein can be implemented as the following aspects, for example.
(1) An information processing device disclosed therein includes a recommended rehabilitation processing unit, an actual-results information acquisition unit, and a nudge processing unit. The recommended rehabilitation processing unit acquires recommended rehabilitation information indicating recommended rehabilitation for a patient suffering from a disease and outputs the recommended rehabilitation in a form perceivable by the patient. The actual-results information acquisition unit acquires rehabilitation actual-results information indicating a status of performance of rehabilitation by the patient. The nudge processing unit executes, based on the recommended rehabilitation information and the rehabilitation actual-results information, nudge processing that prompts performance of rehabilitation that is appropriate to the patient in terms of behavioral economics.

In the information processing device, the nudge processing unit executes the nudge processing that prompts performance of rehabilitation that is appropriate to the patient in terms of behavior economics based on the recommended rehabilitation information indicating the recommended rehabilitation and the rehabilitation actual-results information indicating the status of performance of rehabilitation by the patient. Therefore, the information processing device makes it possible to effectively prompt the patient to perform the appropriate rehabilitation based on the status of performance of rehabilitation by the patient.
(2) The above-described information processing device may have a configuration such that the nudge processing unit performs, as the nudge processing, processing to output nudge information that prompts performance of rehabilitation that is appropriate to the patient in a form perceivable by the patient. With this configuration, it is possible to effectively prompt the patient to perform the appropriate rehabilitation through the output of the nudge information.
(3) The above-described information processing device may have a configuration such that the nudge information includes information that recommends the patient to suppress a rehabilitation performance amount. With this configuration, in a case where the intensity or the frequency of the rehabilitation by the patient is excessive, it is possible to suppress performance of excessive rehabilitation by the patient by recommending the patient to suppress the rehabilitation performance amount.
(4) The above-described information processing device may have a configuration such that a state information acquisition unit that continuously acquires objective state information of the patient and subjective state information based on subjectivity of the patient is further included and the recommended rehabilitation processing unit continuously sets the recommended rehabilitation by using a predetermined information processing method based on the objective state information and the subjective state information. With this configuration, it is possible to set the appropriate recommended rehabilitation in accordance with the daily objective and subjective states of the patient and to effectively prompt the patient to perform the appropriate rehabilitation in accordance with the recommended rehabilitation.
(5) The above-described information processing device may have a configuration such that the objective state information includes an initial analysis result including at least one of a diagnostic estimation result, a progress prediction result, and a therapeutic prediction result executed by another information processing device based on medical examination information at a hospital. With this configuration, it is possible to set more appropriate recommended rehabilitation based on the objective state information including the initial analysis result which is considered to have relatively high accuracy and to effectively prompt the patient to perform the appropriate rehabilitation in accordance with the recommended rehabilitation.
(6) The above-described information processing device may have a configuration such that the objective state information includes biological information of the patient measured using a measurement device on the patient side. With this configuration, it is possible to efficiently acquire the daily objective state information of the patient.
(7) The above-described information processing device may have a configuration such that the subjective state information includes information indicating an answer of the patient to a questionnaire about a Quality of Life (QoL). With this configuration, it is possible to efficiently acquire the daily subjective state information of the patient.
(8) The above-described information processing device may have a configuration such that the disease is a cardiopulmonary disease and the recommended rehabilitation information includes information indicating a target activity amount. With this configuration, it is possible to prompt performance of the appropriate rehabilitation in accordance with the activity amount of the patient.
(9) The above-described information processing device may have a configuration such that the nudge processing unit selects one of a plurality of nudge methods and executes the nudge processing in accordance with the selected nudge method. With this configuration, it is possible to effectively prompt the patient to perform the appropriate rehabilitation.
(10) The above-described information processing device may have a configuration such that the nudge processing unit selects the nudge method based on an attribute of the patient. With this configuration, it is possible to more effectively prompt the patient to perform the appropriate rehabilitation in accordance with the attribute of the patient.
(11) The above-described information processing device may have a configuration such that the attribute includes at least one of age, information on employment, and academic background. With this configuration, it is possible to more effectively prompt the patient to perform the appropriate rehabilitation in accordance with at least one of the age, the information on employment, and the academic background of the patient.
(12) The above-described information processing device may have a configuration such that the nudge processing unit selects the nudge method based on the rehabilitation actual-results information. With this configuration, it is possible to more effectively prompt the patient to perform the appropriate rehabilitation in accordance with the actual results of rehabilitation.
(13) The above-described information processing device may have a configuration such that the rehabilitation actual-results information includes information indicating a status of performance of rehabilitation by the patient after execution of the nudge processing. With this configuration, it is possible to more effectively prompt the patient to perform the appropriate rehabilitation in accordance with the actual results of rehabilitation after the execution of the nudge processing.
(14) The above-described information processing device may have a configuration such that the nudge processing unit selects the nudge method based on a disease condition of the patient. With this configuration, it is possible to more effectively prompt the patient to perform the appropriate rehabilitation in accordance with the disease condition of the patient.

The technique disclosed herein can be implemented in various forms, and for example, can be implemented in the form of an information processing device, an information processing method, a computer program that implements the methods thereof, a non-transitory recording medium in which the computer program is recorded, or the like.

### BRIEF DESCRIPTION OF DRAWINGS

FIG. 1 is an explanatory diagram schematically illustrating a configuration of a medical support system 10 according to the present embodiment.

FIG. 2 is a block diagram schematically illustrating a configuration of a patient terminal device 100.

FIG. 3 is a flowchart illustrating medical support processing.

FIG. 4 is an explanatory diagram illustrating an example of a questionnaire about the QoL.

FIG. 5 is an explanatory diagram illustrating an example of recommended rehabilitation RR.

FIG. 6 is an explanatory diagram illustrating an example of a rehabilitation record image.

FIG. 7 is an explanatory diagram illustrating an example of a nudge method.

FIG. 8 is an explanatory diagram illustrating another example of the nudge method.

### EMBODIMENTS OF THE INVENTION

### A. Embodiment

### A-1. Configuration of Medical Support System 10:

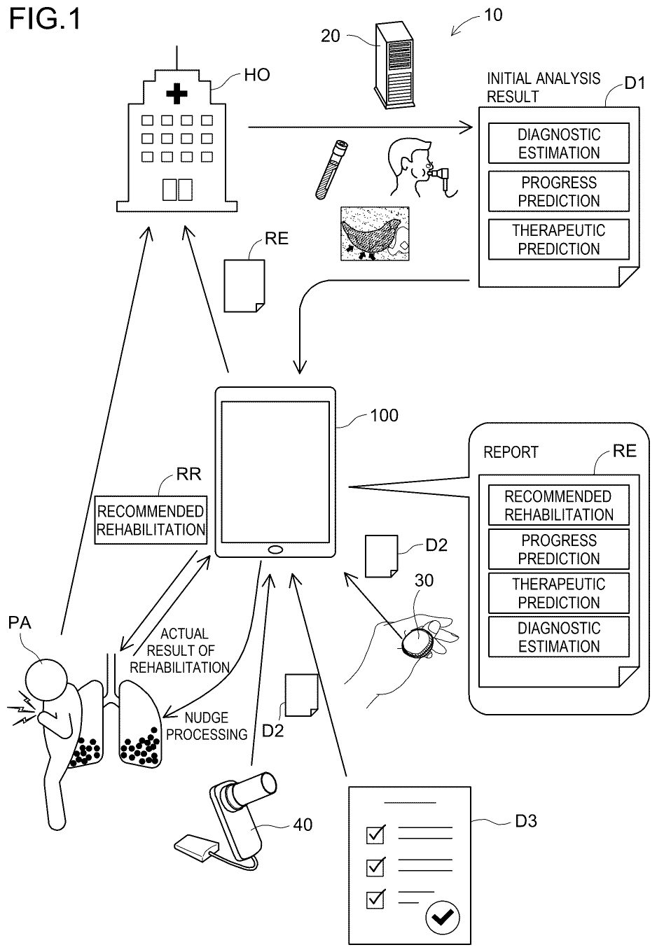

FIG. 1 is an explanatory diagram schematically illustrating a configuration of a medical support system 10 according to the present embodiment. According to the present embodiment, interstitial lung disease is used as a specific example of a disease, and the medical support system 10 that provides a medical support service related to interstitial lung disease will be described. Interstitial lung disease is an example of a cardiopulmonary disease.

The medical support system 10 includes a patient terminal device 100, a server device 20, a wearable device 30, and a lung function measurement device 40. The respective devices constituting the medical support system 10 are communicably connected to one another via a communication network.

The server device 20 is, for example, a device used for diagnosis or the like at a hospital HO. The server device 20 may be installed in the hospital HO or may be installed in another place. For example, the server device 20 executes diagnostic estimation, progress prediction, therapeutic prediction, and the like, using a learned model created by predetermined machine learning based on the medical examination information at the hospital HO and outputs analysis results thereof (hereinafter, referred to as “initial analysis results”). The diagnostic estimation is to estimate the current diseased state, the progress prediction is to predict the future transition of the disease state, and the therapeutic prediction is to predict the effect of therapy (the degree of improvement in the prognosis). The initial analysis result is an example of objective state information indicating an objective state of a patient PA. The server device 20 is an example of another information processing device.

The wearable device 30 is a device that is attached to a predetermined position (e.g., a finger or a wrist) of the body of the patient PA and continuously measures biological information of the patient PA. The wearable device 30 includes, for example, a pulse oximeter and measures the heart rate and the percutaneous oxygen saturation (SpO2). The wearable device 30 may measure other biological information such as a body temperature, a sleeping time, and consumed calories. The biological information measured by the wearable device 30 is an example of objective state information indicating an objective state of the patient PA. The wearable device 30 is an example of a measurement device on the patient side.

The lung function measurement device 40 is a device that is used by the patient PA and measures biological information of the patient PA. The lung function measurement device 40 includes, for example, a spirometer and measures FVC (forced vital capacity) or FEV1 (per-second volume). The lung function measurement device 40 may measure other biological information. The biological information measured by the lung function measurement device 40 is an example of objective state information indicating an objective state of the patient PA. The lung function measurement device 40 is an example of a measurement device on the patient side.

The patient terminal device 100 is a terminal device used by the patient PA suffering from interstitial lung disease. The patient terminal device 100 is, for example, a tablet terminal, a smartphone, or a PC (personal computer).

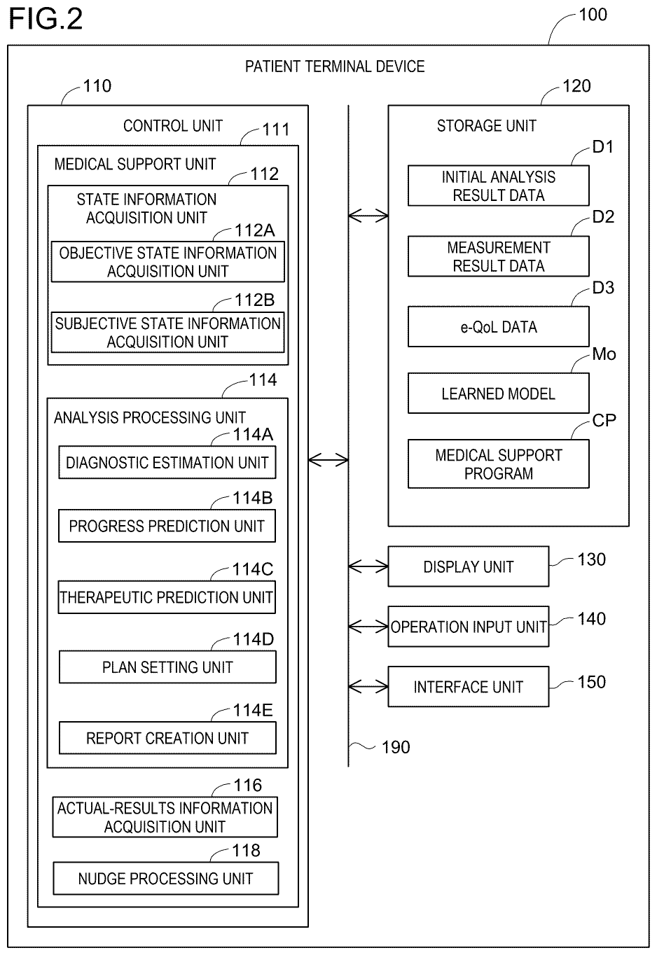

FIG. 2 is a block diagram schematically illustrating a configuration of the patient terminal device 100. The patient terminal device 100 includes a control unit 110, a storage unit 120, a display unit 130, an operation input unit 140, and an interface unit 150. These units are communicably connected to one another via a bus 190. The patient terminal device 100 is an example of an information processing device.

The display unit 130 of the patient terminal device 100 includes, for example, a liquid crystal display and displays various types of images and information. The operation input unit 140 includes, for example, a keyboard, a mouse, a button, and a microphone and receives an operation or an instruction from a user. The display unit 130 may include a touch panel to function as the operation input unit 140. The interface unit 150 includes, for example, a communication interface and performs wired or wireless communications with other devices in accordance with a predetermined communication standard.

The storage unit 120 of the patient terminal device 100 includes, for example, a ROM, a RAM, a hard disk drive (HDD), or a solid state drive (SSD), stores various types of programs and data, and is used as a work area when various programs are executed or a temporary storage area of data. For example, the storage unit 120 stores a medical support program CP that is a computer program for executing various types of processing described below. The medical support program CP is provided in a state of being stored in a computer-readable recording medium (not illustrated) such as a CD-ROM, a DVD-ROM, or a USB memory, or is provided in a state of being able to be acquired from an external device (e.g., a server on a cloud or another terminal device) via the interface unit 150, and is stored in the storage unit 120 in a state of being operable on the patient terminal device 100.

The storage unit 120 of the patient terminal device 100 stores initial analysis result data D1, measurement result data D2, e-QoL data D3, and a learned model Mo in advance or during various types of processing described below. These data and model will be described together with the description of various types of processing described below.

The control unit 110 of the patient terminal device 100 includes, for example, a CPU and controls the operation of the patient terminal device 100 by executing a computer program read from the storage unit 120. For example, the control unit 110 reads the medical support program CP from the storage unit 120 and executes the medical support program CP, thereby functioning as a medical support unit 111 that executes various types of processing described below. The medical support unit 111 includes a state information acquisition unit 112, an analysis processing unit 114, an actual-results information acquisition unit 116, and a nudge processing unit 118. The state information acquisition unit 112 includes an objective state information acquisition unit 112A and a subjective state information acquisition unit 112B. The analysis processing unit 114 includes a diagnostic estimation unit 114A, a progress prediction unit 114B, a therapeutic prediction unit 114C, a plan setting unit 114D, and a report creation unit 114E. The functions of these units will be described together with the description of various type of processing described below.

### A-2. Medical Support Processing:

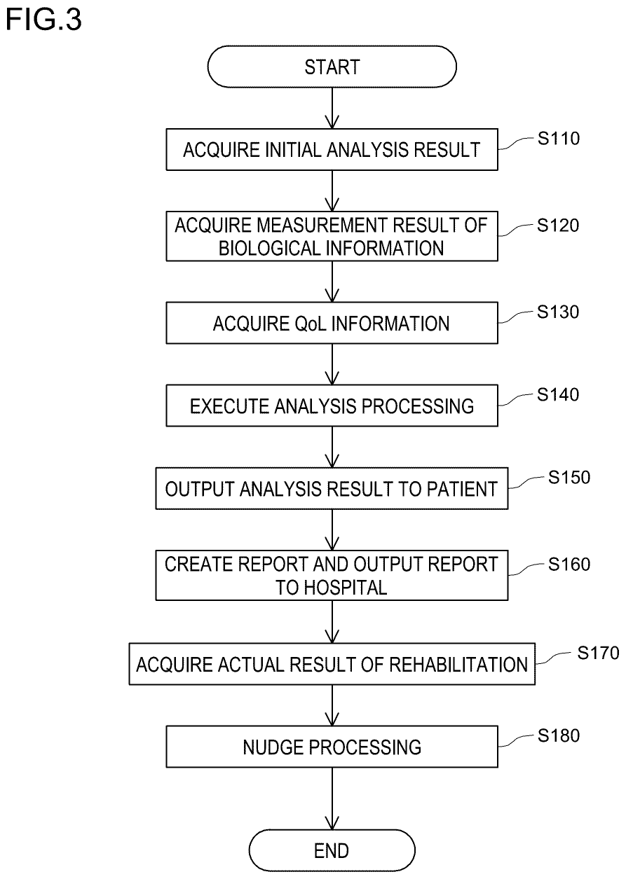

Next, medical support processing executed by the patient terminal device 100 according to the present embodiment will be described. FIG. 3 is a flowchart illustrating the medical support processing. The medical support processing is processing to perform support for improving the symptom of the patient PA suffering from interstitial lung disease. The medical support processing is started, for example, when the patient PA operates the operation input unit 140 of the patient terminal device 100 to input a start instruction.

First, the objective state information acquisition unit 112A (FIG. 2) of the state information acquisition unit 112 of the patient terminal device 100 acquires an initial analysis result (S110). As described above, the initial analysis result is a result of the analysis executed based on the medical examination information of the hospital HO in the server device 20. The initial analysis result includes, for example, at least one of a diagnostic estimation result, a progress prediction result, and a therapeutic prediction result. The objective state information acquisition unit 112A may acquire the initial analysis result by actively accessing the server device 20, or may acquire the initial analysis result by receiving the initial analysis result spontaneously transmitted from the server device 20 to the patient terminal device 100. The initial analysis result data D1 indicating the acquired initial analysis result is stored in the storage unit 120.

The objective state information acquisition unit 112A of the state information acquisition unit 112 of the patient terminal device 100 acquires the biological information of the patient PA measured by the wearable device 30 and/or the lung function measurement device 40 on the patient PA side (S120). The objective state information acquisition unit 112A may acquire the biological information by actively accessing the wearable device 30 and/or the lung function measurement device 40, or may acquire the biological information by receiving the biological information spontaneously transmitted from the wearable device 30 and/or the lung function measurement device 40 to the patient terminal device 100. The acquisition of the biological information is continuously performed (at a frequency of, for example, once a day). The measurement result data D2 indicating the measurement result of the acquired biological information is stored in the storage unit 120.

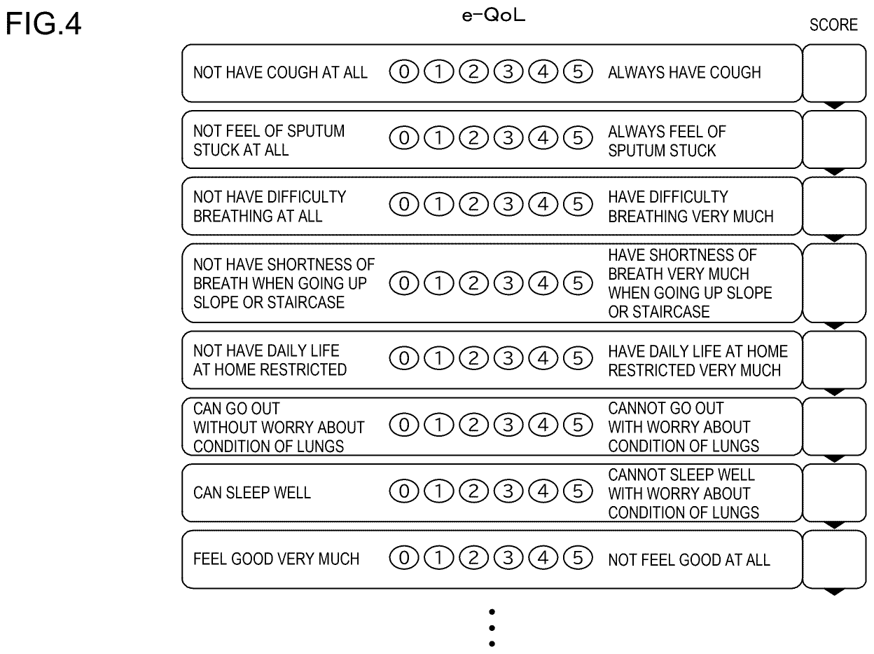

The subjective state information acquisition unit 112B of the state information acquisition unit 112 of the patient terminal device 100 acquires quality of life (QoL) information indicating an answer of the patient PA to a questionnaire about the QoL (S130). To be specific, the subjective state information acquisition unit 112B displays a questionnaire about the QoL of the patient PA on the display unit 130 and acquires the information indicating an answer of the patient PA input via the operation input unit 140. The acquisition of information about the QoL is performed continuously (at a frequency of, for example, once a day). In this specification, the method of electronically acquiring the information about the QoL and the electronically acquired information about the QoL are also referred to as e-QoL. FIG. 4 is an explanatory diagram illustrating an example of a questionnaire about the QoL. The questionnaire about the QoL is for subjective evaluation in six levels, for example, as to whether the person has a cough, feels of sputum stuck, has difficulty breathing, has shortness of breath when going up a slope or a staircase, has daily life at home restricted, cannot go out with worry about the condition of the lungs, cannot sleep with worry about the condition of the lungs, and feels good. The e-QoL data D3 indicating the acquired questionnaire result of the QoL is stored in the storage unit 120.

Subsequently, based on the information acquired from S110 to S130, the analysis processing unit 114 (FIG. 2) of the patient terminal device 100 executes predetermined analysis processing and generates analysis result information indicating a result of the analysis processing (S140). The analysis processing is executed continuously (at a frequency of, for example, once a day). The analysis processing is executed using the learned model Mo created by predetermined machine learning. According to the present embodiment, the analysis processing includes diagnostic estimation, progress prediction, therapeutic prediction, and setting of a plan recommended to be executed by the patient PA for improvement of the symptom of the disease. That is, the diagnostic estimation unit 114A of the analysis processing unit 114 executes diagnostic estimation, the progress prediction unit 114B executes progress prediction, and the therapeutic prediction unit 114C executes therapeutic prediction.

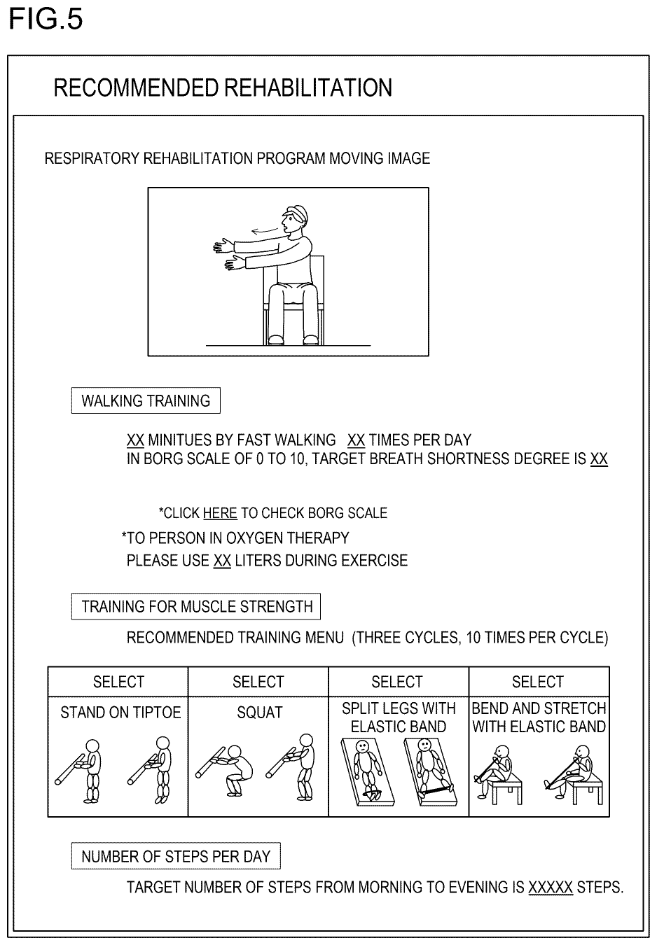

The plan setting unit 114D of the analysis processing unit 114 executes the setting of the plan described above. The plan created by the plan setting unit 114D includes, for example, recommended rehabilitation RR. In this specification, the term “rehab (rehabilitation)” refers to various measures performed for an appropriate life, such as workout, diet, and lifestyle habit re-examination, and leads to, for example, improvement or relief of symptoms themselves, QoL improvement, motor function maintenance and improvement, life prognosis prolongation, and the like. FIG. 5 is an explanatory diagram illustrating an example of the recommended rehabilitation RR. The recommended rehabilitation RR is a menu (exercise, intensity, and the like) of rehabilitation recommended to be performed in accordance with the state of the individual patient PA. A target activity amount may be set as the recommended rehabilitation RR. The recommended rehabilitation information indicating the recommended rehabilitation RR is included in the above-described analysis result information.

The plan created by the plan setting unit 114D may include a visit to a hospital. For example, in consideration of the state of the patient PA, in a case where the improvement of the symptom of the disease is not expected or the worsening of the symptom is predicted, the plan setting unit 114D may set a visit to a hospital as a plan recommended to be executed by the patient PA for the improvement of the symptom of the disease.

The analysis processing unit 114 outputs the result of the analysis processing in a form perceivable by the patient PA based on the above-described analysis result information (S150). For example, as illustrated in FIG. 5, the plan setting unit 114D of the analysis processing unit 114 displays the recommended rehabilitation RR on the display unit 130 by using characters or images (still images or moving images) based on the recommended rehabilitation information described above. Thus, the patient PA can recognize the rehabilitation menu recommended to be executed. The plan setting unit 114D is an example of a recommended rehabilitation processing unit.

For example, the plan setting unit 114D of the analysis processing unit 114 may display the message that recommends a visit to the hospital on the display unit 130. This makes it possible to prompt the patient PA to visit the hospital. The analysis processing unit 114 may output another analysis result. For example, the analysis processing unit 114 may display the result of the progress prediction on the display unit 130. Thus, the patient PA can grasp the progress prediction of the disease condition.

The report creation unit 114E of the analysis processing unit 114 creates a report RE in which the results of the analysis processing are summarized based on the above-described analysis result information and outputs the report RE to the hospital HO (S160). Thus, a doctor or the like in the hospital HO can grasp the daily objective and subjective states of the patient PA and the results of the diagnostic estimation, the progress prediction, and the therapeutic prediction based on the states, and can use the results for the therapy of the patient PA. Among the analysis results, the results of the diagnostic estimation and the therapeutic prediction may be output only to the hospital HO without being output to the patient PA.

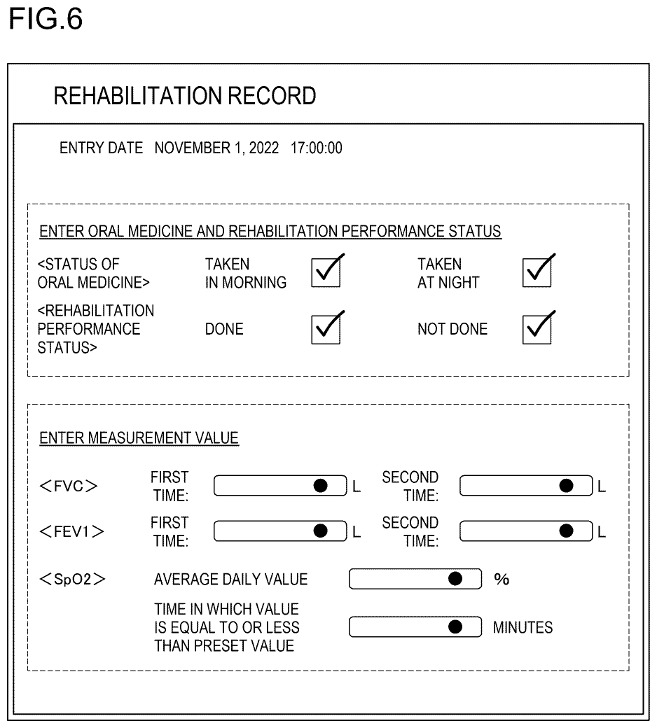

The actual-results information acquisition unit 116 (FIG. 2) of the patient terminal device 100 acquires rehabilitation actual-results information indicating the status of performance of rehabilitation by the patient PA (S170). The rehabilitation actual-results information is information indicating the status of performance (performance date and time or performance content) of rehabilitation by the patient PA. For example, the actual-results information acquisition unit 116 may display the rehabilitation record image illustrated in FIG. 6 on the display unit 130 and acquire the rehabilitation actual-results information based on the content input by the patient PA via the operation input unit 140. Alternatively, in a case where the moving image indicating the recommended rehabilitation RR is reproduced by the operation of the patient PA, the actual-results information acquisition unit 116 may acquire the rehabilitation actual-results information indicating that the rehabilitation has been performed by the patient PA. Since it is expected that there is a change in the biological information (e.g., heart rate or SpO₂) of the patient PA accompanied with the performance of the rehabilitation, the actual-results information acquisition unit 116 may acquire the rehabilitation actual-results information indicating that the rehabilitation has been performed by the patient PA when there is a predetermined change in the biological information measured by the wearable device 30 or the like. For example, in a case where the event indicating that the patient PA has performed rehabilitation is not detected by a predetermined determination timing, the actual-results information acquisition unit 116 may acquire the rehabilitation actual-results information indicating that the patient PA has not performed rehabilitation by the determination timing.

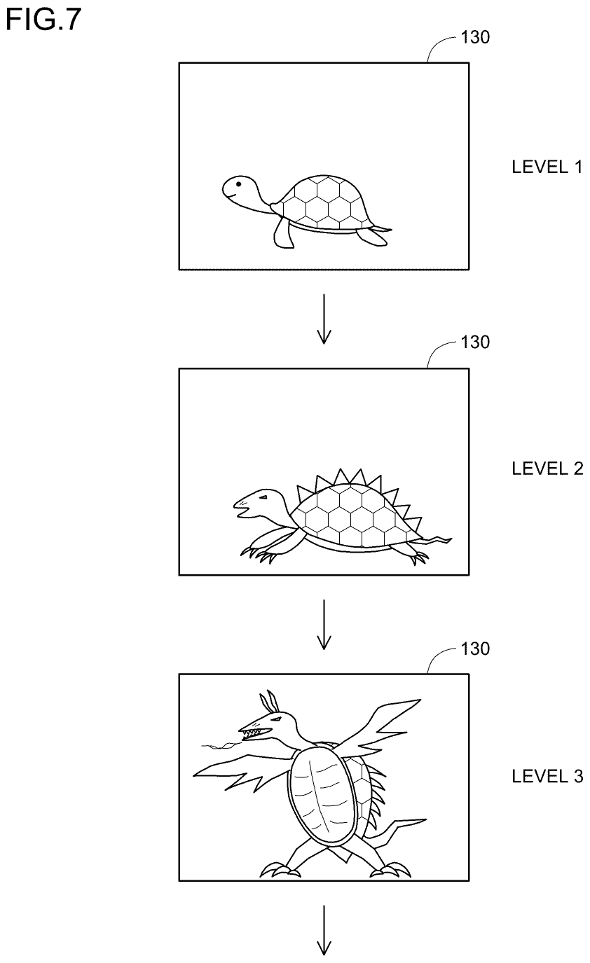

The nudge processing unit 118 (FIG. 2) of the patient terminal device 100 executes nudge processing based on the recommended rehabilitation information and the rehabilitation actual-results information (S180). In this specification, “nudge” refers to a mechanism serving as a factor for prompting the action of the user from the viewpoint of behavioral economics. According to the present embodiment, the user in the nudge is a patient, and the behavior is rehabilitation. The nudge processing is processing that prompts performance of rehabilitation that is appropriate to the patient PA in terms of behavioral economics (that is, in accordance with the recommended rehabilitation RR). Prompting the patient PA to perform rehabilitation in terms of behavioral economics includes motivating the patient PA to perform rehabilitation by operating the reward system of the patient PA. For example, the nudge processing unit 118 outputs the nudge information that prompts performance of rehabilitation that is appropriate to the patient PA in a form perceivable by the patient PA. As illustrated in FIG. 7, the nudge processing unit 118 may perform nudge processing using what is called a gamification method. In the example of FIG. 7, each time the patient PA performs rehabilitation in accordance with the recommended rehabilitation RR, the level of the character displayed on the display unit 130 is increased, and the character grows to a strong character. Accordingly, it is expected that the patient PA actively engages in the rehabilitation with motivation to grow the character displayed on the display unit 130.

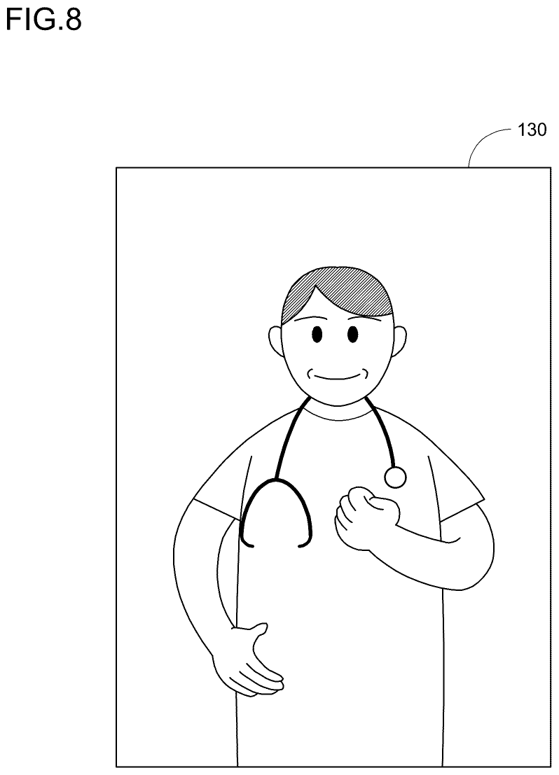

Alternatively, as illustrated in FIG. 8, the nudge processing unit 118 may cause the display unit 130 to display the moving image in which the doctor in charge praises the patient PA each time the patient PA performs rehabilitation in accordance with the recommended rehabilitation RR. Accordingly, it is expected that the patient PA actively engages in the rehabilitation with the motivation to be praised by the doctor in charge displayed on the display unit 130. On the other hand, in a case where the patient PA does not perform the rehabilitation in accordance with the recommended rehabilitation RR, the nudge processing unit 118 may cause the display unit 130 to display the moving image in which the doctor in charge tells the importance of the rehabilitation or the moving image in which the doctor in charge encourages the patient PA.

In a case where the patient PA performs an excessive amount of rehabilitation as compared with the recommended rehabilitation RR, the nudge processing unit 118 may cause the display unit 130 to display the image that recommends the patient PA to suppress the rehabilitation performance amount. It may be determined whether the amount of rehabilitation is excessive depending on, for example, whether the amount of rehabilitation exceeds a target activity amount set for the recommended rehabilitation RR, or based on a change in the biological information of the patient PA. In each of the above-described examples, the image displayed on the display unit 130 is an example of the nudge information.

The following methods are also considered as the nudge method.
Method for displaying the information emphasizing rehabilitation effects and short-term lost profits
Method for performing conversations by name using a chatbot or the like
Method for explaining that data is fed back to the doctor in charge, or the like
Method using a mechanism that allows acquisition of a stamp or the like by performing rehabilitation
Method using the peer effect. For example, the method for visualizing the degree of diligence in rehabilitation of patients with the same disease and the same severity by using the behavioral characteristics that the patient can be diligent in the presence of a fellow.
Method using social preference. For example, the method for visualizing the enjoyment of the doctor or the family according to a rehabilitation performance status by using behavioral characteristics of the desire to distinguish the other person when there is the other person.
Method using reciprocity. For example, the method using a mechanism in which a contribution is made to the study of the disease in accordance with the diligence in rehabilitation by using the behavioral characteristics in which a person feels that he/she has to return when receiving something.
Method using the Simulacrum effect. For example, the method for increasing sympathy by displaying a delighted expression on the face of the doctor in charge or the family on a UI screen.
Method using authority. For example, the method describing expert support or using citations.
Method using the band wagon effect. For example, the method for ranking and displaying popular rehabilitation trainings by using the behavioral characteristics of the desire to participate where the people gather.
Method using the herding effect. For example, the method for displaying a message such as “Are you OK with everyone performing rehabilitation?” by using the behavioral characteristics in which the minority feels anxious.
Method using the Nash equilibrium. For example, the method for increasing the doctor ranking of the doctor in charge in accordance with the patient's diligence in rehabilitation and decreasing the doctor ranking when the patient stops the rehabilitation by using the behavioral characteristics that the patient cannot escape. It can be changed to a favorite doctor or character.
Method using rarity. For example, the method in which the account is deleted when no rehabilitation is performed for a certain period of time and there is a need to make excuses to the doctor by using the behavioral characteristics that a person wants it when he/she almost loses it. For example, the method for increasing the point awarding rate only for a limited period.
Method using social proof. For example, the method for indicating that the life of a person who is engaged in rehabilitation is extended or the rehabilitation training selected from data so far is suitable for the patient by using the behavioral characteristics that the person wants to rely on something. The method for telling the reason that it is fine even if it is hard.
Method using the bystander effect. For example, the method of not acting independently in a group but acting as a group (speaking by name). For example, the method in which formality is first adopted and casualness is gradually adopted.
Method using heuristic. For example, the method for putting a familiar element in a UI, using an intuitive UI, or using a similar questionnaire by using a mental shortcut or behavioral characteristics of immediate conclusion.
Method using the present bias. For example, the method for indicating a specific index of target achievement in a period as short as possible (including the number of times) or displaying an outcome for each predetermined period (e.g., one week) by using a tendency of overestimating a profit obtained most recently more than a larger profit obtained in the future.

According to the present embodiment, the nudge processing unit 118 selects one of a plurality of nudge methods and executes the nudge processing in accordance with the selected nudge method. Specifically, the nudge processing unit 118 selects a nudge method based on the attribute of the patient PA. For example, the nudge processing unit 118 refers to the age of the patient PA and, when the patient PA is a young person, selects the nudge method of gamification illustrated in FIG. 7, and, when the patient PA is an elderly person, selects the nudge method using the image of the doctor in charge illustrated in FIG. 8. The attribute of the patient PA to be referred to is not limited to the age of the patient PA, and for example, other attributes such as information on employment (occupation, type of employment, annual income, and the like) and academic background may be referred to.

Alternatively, the nudge processing unit 118 selects the nudge method based on the rehabilitation actual-results information. For example, in a case where the patient PA did not perform rehabilitation regardless of first nudge processing performed by a predetermined nudge method (e.g., the method using the moving image in which the doctor in charge tells the importance of rehabilitation), the nudge processing unit 118 selects, as the method of the subsequent nudge processing to be executed, a nudge method (e.g., the method using the moving image in which the doctor in charge encourages the patient PA) which is considered to have a higher effect of prompting the performance of rehabilitation. As described above, the nudge processing unit 118 selects the method of the subsequent nudge processing to be executed based on the information indicating the status of performance of the rehabilitation by the patient PA after the execution of the nudge processing by the predetermined nudge method as the rehabilitation actual-results information. The rehabilitation actual-results information to be referred to is not limited to the response of the patient PA to the nudge processing described above and, for example, the simple information indicating the status of performance of rehabilitation in which the influence of the nudge processing is not considered may be referred to.

The nudge processing unit 118 may select the nudge method based on the disease condition (e.g., severity) of the patient PA. For example, the nudge processing unit 118 may select the nudge method of gamification described above for the patient PA with relatively moderate disease and may select the method using the moving image, in which the doctor in charge encourages the patient PA as described above, for the patient PA with relatively severe disease.

As described above, the nudge processing unit 118 executes the nudge processing that prompts performance of rehabilitation that is appropriate to the patient PA in terms of behavioral economics so that it is expected that the patient PA actively engages in daily rehabilitation and further it is expected that the prognosis of the patient PA and the QoL are improved. At least a part of the process from S120 to S180 may be repeatedly executed. Repeated execution as described above improves the accuracy of the analysis processing using the learned model Mo and improves the usefulness of the patient terminal device 100.

### A-3. Effect of Present Embodiment:

As described above, the patient terminal device 100 according to the present embodiment includes the plan setting unit 114D, the actual-results information acquisition unit 116, and the nudge processing unit 118. The plan setting unit 114D acquires the recommended rehabilitation information indicating the recommended rehabilitation RR for the patient PA suffering from the disease and outputs the recommended rehabilitation RR in a form perceivable by the patient PA. The actual-results information acquisition unit 116 acquires the rehabilitation actual-results information indicating the status of performance of rehabilitation by the patient PA. Based on the recommended rehabilitation information and the rehabilitation actual-results information, the nudge processing unit 118 performs the nudge processing that prompts performance of rehabilitation that is appropriate to the patient PA in terms of behavioral economics.

As described above, in the patient terminal device 100 according to the present embodiment, the nudge processing unit 118 executes the nudge processing that prompts performance of rehabilitation that is appropriate to the patient PA in terms of behavioral economics based on the recommended rehabilitation information indicating the recommended rehabilitation RR for the patient PA and the rehabilitation actual-results information indicating the status of performance of the rehabilitation by the patient PA. Therefore, the patient terminal device 100 according to the present embodiment makes it possible to effectively prompt the patient PA to perform the appropriate rehabilitation in accordance with the recommended rehabilitation RR in consideration of the status of performance of the rehabilitation by the patient PA.

In the patient terminal device 100 according to the present embodiment, the nudge processing unit 118 performs, as the nudge processing, the processing to output the nudge information that prompts performance of the rehabilitation that is appropriate to the patient PA in a form perceivable by the patient PA. Therefore, the patient terminal device 100 according to the present embodiment makes it possible to effectively prompt the patient PA to perform the appropriate rehabilitation through the output of the nudge information.

In the patient terminal device 100 according to the present embodiment, the nudge information includes the information that recommends the patient PA to suppress the rehabilitation performance amount. Therefore, in a case where the intensity or the frequency of the rehabilitation by the patient PA is excessive, the patient terminal device 100 according to the present embodiment makes it possible to suppress performance of excessive rehabilitation by the patient PA by recommending the patient PA to suppress the rehabilitation performance amount.

The patient terminal device 100 according to the present embodiment further includes the state information acquisition unit 112 that continuously acquires the objective state information of the patient PA and the subjective state information based on the subjectivity of the patient PA. The plan setting unit 114D continuously sets the recommended rehabilitation RR by using a predetermined information processing method based on the objective state information and the subjective state information. Therefore, the patient terminal device 100 according to the present embodiment makes it possible to set the appropriate recommended rehabilitation RR in accordance with the daily objective and subjective states of the patient PA and to effectively prompt the patient PA to perform the appropriate rehabilitation in accordance with the recommended rehabilitation RR.

In the patient terminal device 100 according to the present embodiment, the objective state information includes the initial analysis result including at least one of a diagnostic estimation result, a progress prediction result, and a therapeutic prediction result executed by another information processing device based on the medical examination information at the hospital HO. Therefore, the patient terminal device 100 according to the present embodiment makes it possible to set more appropriate recommended rehabilitation RR based on the objective state information including the initial analysis result, which is considered to have relatively high accuracy and to effectively prompt the patient PA to perform the appropriate rehabilitation in accordance with the recommended rehabilitation RR.

In the patient terminal device 100 according to the present embodiment, the objective state information includes the biological information of the patient PA measured by using a measurement device (e.g., the wearable device 30 and/or the lung function measurement device 40) on the patient PA side. Therefore, the patient terminal device 100 according to the present embodiment makes it possible to efficiently acquire the daily objective state information of the patient PA.

In the patient terminal device 100 according to the present embodiment, the subjective state information includes the information indicating an answer of the patient to the questionnaire about the QoL. Therefore, the patient terminal device 100 according to the present embodiment makes it possible to efficiently acquire the daily subjective state information of the patient PA.

In the patient terminal device 100 according to the present embodiment, the disease is a cardiopulmonary disease, and the recommended rehabilitation information includes the information indicating a target activity amount. Therefore, the patient terminal device 100 according to the present embodiment makes it possible to prompt performance of the appropriate rehabilitation in accordance with the activity amount of the patient PA.

In the patient terminal device 100 according to the present embodiment, the nudge processing unit 118 selects one of the plurality of nudge methods and executes the nudge processing in accordance with the selected nudge method. Therefore, the patient terminal device 100 according to the present embodiment makes it possible to effectively prompt the patient PA to perform the appropriate rehabilitation. The nudge processing unit 118 may select a nudge method based on the attribute of the patient PA. In this way, it is possible to more effectively prompt the patient PA to perform the appropriate rehabilitation in accordance with the attribute of the patient. The nudge processing unit 118 may select a nudge method based on the rehabilitation actual-results information. In this way, it is possible to more effectively prompt the patient PA to perform the appropriate rehabilitation in accordance with the actual results of rehabilitation.

### B. Modification

The technique disclosed herein is not limited to the above-described embodiment and can be modified into various forms without departing from the gist thereof, and for example, the following modifications are also possible.

The configuration of the medical support system 10 according to the above-described embodiment is merely an example, and can be variously modified. For example, according to the above-described embodiment, the medical support system 10 includes the server device 20, the wearable device 30, and the lung function measurement device 40, but the medical support system 10 may not include at least one of these devices, or the medical support system 10 may include a device other than these devices.

The content of the medical support processing according to the above embodiment is merely an example, and various modifications are possible. For example, the objective state information and the subjective state information used in the above embodiment are merely examples, and other information may be used.

According to the above-described embodiment, the recommended rehabilitation is set based on the objective state information and the subjective state information, but the recommended rehabilitation may be set without using at least one of the objective state information and the subjective state information. According to the above-described embodiment, the patient terminal device 100 sets the recommended rehabilitation, but another device (e.g., the server device 20) may set the recommended rehabilitation, and the patient terminal device 100 may acquire the information indicating the set recommended rehabilitation.

According to the above-described embodiment, the subjective state information of the patient PA is acquired using the questionnaire illustrated in FIG. 4, but the acquisition form of the subjective state information can be arbitrarily changed.

According to the above-described embodiment, the recommended rehabilitation RR is displayed in the form illustrated in FIG. 5, but the display form of the recommended rehabilitation RR can be arbitrarily changed. The output form of the recommended rehabilitation RR is not limited to image display, and may be another form such as audio output.

According to the above-described embodiment, the rehabilitation actual-results information indicating the status of performance of the rehabilitation by the patient PA is acquired using the rehabilitation record image illustrated in FIG. 6, but the acquisition form of the rehabilitation actual-results information can be arbitrarily changed.

According to the above-described embodiment, the analysis processing is executed using the learned model Mo created by the predetermined machine learning, but the analysis processing may be executed using another information processing method.

According to the above-described embodiment, the patient terminal device 100 is exemplified as an information processing device, but the information processing device to which the technique disclosed herein is applicable is not limited to the patient terminal device 100, and may be, for example, a device installed in the hospital HO. For example, the technique disclosed herein may be applied to a measurement device such as the lung function measurement device 40.

According to the above-described embodiment, the information processing for supporting the therapy of the patient PA suffering from interstitial lung disease is exemplified, but the technique disclosed herein is not limited to interstitial lung disease and can be similarly applied to the information processing for supporting the therapy of the patient PA suffering from a disease.

According to the above-described embodiment, a part of the configuration implemented by hardware may be replaced with software, and conversely, a part of the configuration implemented by software may be replaced with hardware.

### DESCRIPTION OF REFERENCE NUMERALS

10: MEDICAL SUPPORT SYSTEM
20: SERVER DEVICE
30: WEARABLE DEVICE
40: LUNG FUNCTION MEASUREMENT DEVICE
100: PATIENT TERMINAL DEVICE
110: CONTROL UNIT
111: MEDICAL SUPPORT UNIT
112: STATE INFORMATION ACQUISITION UNIT
112A: OBJECTIVE STATE INFORMATION ACQUISITION UNIT
112B: SUBJECTIVE STATE INFORMATION ACQUISITION UNIT
114: ANALYSIS PROCESSING UNIT
114A: DIAGNOSTIC ESTIMATION UNIT
114B: PROGRESS PREDICTION UNIT
114C: THERAPEUTIC PREDICTION UNIT
114D: PLAN SETTING UNIT
114E: REPORT CREATION UNIT
116: ACTUAL-RESULTS INFORMATION ACQUISITION UNIT
118: NUDGE PROCESSING UNIT
120: STORAGE UNIT
130: DISPLAY UNIT
140: OPERATION INPUT UNIT
150: INTERFACE UNIT
190: BUS

1. An information processing device comprising:
a recommended rehabilitation processing unit that acquires recommended rehabilitation information indicating recommended rehabilitation for a patient suffering from a disease and outputs the recommended rehabilitation in a form perceivable by the patient;
an actual-results information acquisition unit that acquires rehabilitation actual-results information indicating a status of performance of rehabilitation by the patient; and
a nudge processing unit that executes, based on the recommended rehabilitation information and the rehabilitation actual-results information, nudge processing that prompts performance of rehabilitation that is appropriate to the patient in terms of behavioral economics.

2. The information processing device according to claim 1, wherein the nudge processing unit performs, as the nudge processing, processing to output nudge information that prompts performance of rehabilitation that is appropriate to the patient in a form perceivable by the patient.

3. The information processing device according to claim 2, wherein the nudge information includes information that recommends the patient to suppress a rehabilitation performance amount.

4. The information processing device according to claim 1, further comprising a state information acquisition unit that continuously acquires objective state information of the patient and subjective state information based on subjectivity of the patient, wherein
the recommended rehabilitation processing unit continuously sets the recommended rehabilitation by using a predetermined information processing method based on the objective state information and the subjective state information.

5. The information processing device according to claim 4, wherein the objective state information includes an initial analysis result including at least one of a diagnostic estimation result, a progress prediction result, and a therapeutic prediction result executed by another information processing device based on medical examination information at a hospital.

6. The information processing device according to claim 4, wherein the objective state information includes biological information of the patient measured using a measurement device on the patient side.

7. The information processing device according to claim 4, wherein the subjective state information includes information indicating an answer of the patient to a questionnaire about a Quality of Life (QoL).

8. The information processing device according to claim 1, wherein
the disease is a cardiopulmonary disease, and
the recommended rehabilitation information includes information indicating a target activity amount.

9. The information processing device according to claim 1, wherein the nudge processing unit selects one of a plurality of nudge methods and executes the nudge processing in accordance with the selected nudge method.

10. The information processing device according to claim 9, wherein the nudge processing unit selects the nudge method based on an attribute of the patient.

11. The information processing device according to claim 10, wherein the attribute includes at least one of age, information on employment, and academic background.

12. The information processing device according to claim 9, wherein the nudge processing unit selects the nudge method based on the rehabilitation actual-results information.

13. The information processing device according to claim 12, wherein the rehabilitation actual-results information includes information indicating a status of performance of rehabilitation by the patient after execution of the nudge processing.

14. The information processing device according to claim 9, wherein the nudge processing unit selects the nudge method based on a disease condition of the patient.

15. An information processing method comprising:
a step of acquiring recommended rehabilitation information indicating recommended rehabilitation for a patient suffering from a disease and outputting the recommended rehabilitation in a form perceivable by the patient;
a step of acquiring rehabilitation actual-results information indicating a status of performance of rehabilitation by the patient; and
a step of executing, based on the recommended rehabilitation information and the rehabilitation actual-results information, nudge processing that prompts performance of rehabilitation that is appropriate to the patient in terms of behavioral economics.

16. A non-transitory recording medium storing a computer program causing a computer to execute:
processing to acquire recommended rehabilitation information indicating recommended rehabilitation for a patient suffering from a disease and output the recommended rehabilitation in a form perceivable by the patient;
processing to acquire rehabilitation actual-results information indicating a status of performance of rehabilitation by the patient; and
based on the recommended rehabilitation information and the rehabilitation actual-results information, nudge processing that prompts performance of rehabilitation that is appropriate to the patient in terms of behavioral economics.
